(19) United States
(12) Patent Application Publication
(10) Pub. No.: US 2015/0065375 A1
(43) Pub. Date: Mar. 5, 2015

(54) ACQUIRED IMMUNITY BIOMARKERS AND USES THEREOF

(71) Applicant: ISTITUTO NAZIONALE DI GENETICA MOLECOLARE - INGM, Milano (MI) (IT)

(72) Inventors: Sergio Abrignani, Serre di Rapolano (SI) (IT); Paola De Candia, Milano (MI) (IT)

(21) Appl. No.: 14/394,822

(22) PCT Filed: Apr. 10, 2013
(86) PCT No.: PCT/EP2013/057453

§ 371 Date: Oct. 16, 2014

(30) Foreign Application Priority Data
Apr. 17, 2012 (EP) 12164436.3

(51) Int. Cl. C12Q 1/68

(52) U.S. Cl. CPC C12Q 1/6883; C12Q 2600/178; C12Q 2600/158
USPC 506/9; 435/6.12; 435/6.11; 506/16

(57) ABSTRACT

The present invention relates to a biomarker of immunity response for use in monitoring the acquired immunity of an immunized subject, to an in vitro method and a kit for monitoring the acquired immunity of an immunized subject.

### FIELD OF THE INVENTION

MicroRNAs (miRNAs) have pivotal role in immune cells differentiation and function. It has been shown that communication between immune cells involves not only secretion of cytokines and chemokines, but also the release of membrane vesicles, that enclose soluble cellular components, including miRNAs.

The authors have previously shown that miR-150 and miR-19b are strongly expressed in human resting lymphocytes, with highest levels in CD4 cells [Rossi et al., 2011]; now the authors have found that they are highly associated to nanovesicles purified from the extracellular milieu of in vitro activated lymphocytes. Notably, nanovesicles-associated miR-150 [ID: has-miR-150-5p; Accession MIMAT0000451; Sequence: ucucccaacccuuguaccagug (SEQ ID No. 1)] and miR-19b [ID: has-miR-19b-3p; Accession: MIMAT0000074; Sequence: ugugcaaauccaugcaaaacuga (SEQ ID No. 2)] did consistently rise in serum of adults upon immunization with a single dose of MF-59 adjuvanted H1N1 pandemic flu vaccine, whereas no significant increase was detectable when analyzing purified microvesicle-associated miRNAs, suggesting a specific releasing process that results in a long-range exchange involving miRNAs upon physiological activation of the immune system.

The substantial release of specific miRNAs by lymphocytes is a phenomenon with still unrecognized functional role of induction, amplification and modulation of immune responses. Moreover, the present results open up the possibility of using nanovesicle-associated miRNAs as novel biomarkers of immunity.

### STATE OF THE ART

miRNAs represent a class of small RNAs, 18-25 nucleotides in length, that regulate gene expression in a post-transcriptional way, via sequence-specific interactions usually to the 3′UTR of mRNA target sites (Bartel 2004). miRNAs are known to be present across most species and are very highly conserved. About 2000 miRNAs have been described in the human transcriptome (as for miRBase, Release 19 http://microrna.sanger.ac.uk/) and they are assumed to regulate the majority of human genes (Friedman, Farh et al. 2009). Large amount of miRNAs derived from various tissues/organs are present in human blood and circulate in a cell-free and stable form that is protected from endogenous RNase attack (Chen, Ba et al. 2008; Mitchell, Parkin et al. 2008). The dominant model to explain the stability of circulating miRNAs was that they could be released from cells in membrane-bound vesicles, which would be the reason why they are protected from blood RNase activity. More recently it has also been proposed that the majority of circulating miRNAs could be associated with proteins, and that the preferential association of circulating miRNAs to different biological structures (based on proteic complexes versus lipidic membranes) could be dictated by the preferential releasing process of the originating tissue (Arroyo, Chevillet et al. 2011). Vesicles proposed as carriers for the circulating miRNAs include large membrane-surrounded bodies as large as 1 μm, presumably formed through budding/blebbing of the plasma membrane and generally defined as microvesicles, senescent and apoptotic bodies of similar size. A more specific class of circulating vesicles is constituted by exosomes, which are 20- to 100-nm in size (hence generically defined as nano-sized vesicles or nanovesicles), are released through the intracellular membrane fusion of multivesicular bodies with the plasma membrane, and have fusogenic activity. Exosomes are released by most cell types and are now widely recognized as conveyors of intercellular communication (Simons and Raposo 2009), as it is the case when dendritic cells internalize exosomes with specific MHC-peptide complexes and in so doing aquire new antigen presenting specificities. (Raposo, Nijman et al. 1996). More recently, the finding that exosomes carry genetic materials, including miRNAs, has been a major breakthrough, revealing their capacity to vehicle genetic messages (Valadi, Ekstrom et al. 2007) although the role of miRNAs released in exosomes is still poorly known (Thery, Ostrowski et al. 2009).

miRNA expression change specifically in diseases such as cancer, autoimmunity and viral infections (Jopling, Yi et al. 2005; Volinia, Calin et al. 2006; O'Connell, Taganov et al. 2007), and the description of disease-associated miRNA signatures makes this class of molecules a possible new class of blood-based non-invasive biomarkers (Chen, Ba et al. 2008). It has been shown that circulating miRNA profiles can discriminate healthy subjects from patients affected by cardiovascular diseases, multiple sclerosis, sepsis, liver injury, different tumor types, as well as physiological states, such as pregnancy (Reid, Kirschner et al. 2011, Chim, Shing et al. 2008; Wang, Yu et al. 2010; Bala, Petrasek et al. 2012).

There is still the need to address whether serum circulating nanovesicles may contain a footprint of a substantial and tangible release of specific miRNAs by immune cells during immune response, a phenomenon with still unrecognized functional role. Moreover, there is the need of identifying a signature of serum circulating miRNAs that could be used as valuable non-invasive biomarkers of immune response.

WO2011/158191 refers to detecting miRNA expression profiles to monitoring the immune system of a subject. Though vaccination is mentioned in the document, no data supporting the modulation of specific miRNA in vaccinated people are present, nor of the specific miRNAs of the present invention.

### DESCRIPTION OF THE INVENTION

Authors have here characterized total miRNome (genome-wide miRNA expression profile) associated to circulating nanovesicles and how it is affected by a physiological immune response, as the one elicited by vaccination. Authors have found miR-150 and/or miR19b to be strongly associated to circulating exosomes in human serum and to specifically increase upon antigenic challenge with pandemic flu vaccine both in adults and children. miR-150 was also shown to increase specifically in sera of children who acquired immunity to varicella upon vaccination but not in sera of children who did not.

Communication between cells of the immune system involves not only secretion of proteins, such as cytokines and chemokines, but also the release of membrane vesicles, that enclose soluble components of cellular origin, including proteins and microRNAs (miRNA). The functional consequences of vesicle transfer can theoretically be the induction, amplification and modulation of immune responses.

Authors' hypothesis is that there exists a productive exchange involving miRNA containing vesicles released by recently activated cells of the immune system. To verify this hypothesis authors first analyzed the association of miRNAs with nano-sized vesicles circulating in the bloodstream of healthy donors.

In order to gain more insight into extracellular miRNAs compartmentalization, authors have also implemented a new filtration-based process that allows the purification of RNA contained in either nanovesicles (i.e. exosomes, 20 to 200 nm) or microvesicles (i.e. microparticles, apoptotic and senescent bodies, larger than 200 nm) starting from very low volume of sera or cell conditioned medium.

Authors had previously shown that miR-150 and miR-19b are distinctly expressed in human resting lymphocytes, with highest levels in CD4 cells [Rossi et al., 2011]. Now authors have found that miR-150 and miR-19b are highly associated with nanovesicles purified from the extracellular milieu of lymphocytes upon activation. Notably, authors have then been able to show that nanovesicle-associated miR-150 and miR-19b did consistently increase upon vaccination, whereas no significant difference was detectable when analyzing purified microvesicle-associated miRNAs, suggesting a miRNA releasing process that results in compartmentalization of these miRNAs in nanovescicles.

Therefore, the specific rise of miR-150 and miR-19b that authors observe in sera of vaccinated individuals 30 days after immunization may be a footprint of a substantial release of specific miRNA containing nanovesicles by CD4 T cells and/or B cells during immune response, a phenomenon with still unrecognized functional role.

Therefore an object of the invention is a biomarker consisting of at least one miRNA selected between miR-150 (SEQ ID No. 1) and miR-19b (SEQ ID No. 2), for use in monitoring the acquired immunity of an immunized subject in an isolated biological sample.

In a preferred embodiment of the invention said biomarker consists of miR-150 (SEQ ID No. 1) and miR-19b (SEQ ID No. 2).

Said biological sample is preferably serum or cellular medium of ex vivo cultured cells of the immunized subject.

Said miRNA is preferably associated to nanovesicles isolated from the biological sample of the immunized subject

In a preferred embodiment, the acquired immunity is due to a vaccination, more preferably a flu or varicella vaccination.

Even more preferably said flu vaccination is performed with the H1N1 MF59 vaccine.

Even more preferably said varicella vaccination is performed with Measles-Mumps-Rubella-Varicella vaccine.

Another object of the invention is an in vitro method of monitoring the acquired immunity of an immunized subject comprising the following steps:

a) measuring the amount of the biomarker as above described in a biological sample isolated from the immunized subject, and
b) comparing the measured amount of step a) with an appropriate control amount of said biomarker, wherein if the amount of said biomarker in the biological sample is higher than the control amount, this indicates that the immunized subject is effectively protected.

The subject can be an adult or a child. The biological sample is preferably a biological fluid as blood, plasma serum or cellular medium of ex vivo cultured cells of the immunized subject, more preferably nanovesicles extracted from the biological sample.

In a preferred embodiment, the acquired immunity is due to a vaccination, more preferably a flu or varicella vaccination.

Even more preferably, said flu vaccination is performed with the H1N1 MF59 vaccine.

Even more preferably, said varicella vaccination is performed with Measles-Mumps-Rubella-Varicella vaccine.

The amount of the biomarker is preferably measured by specific acid nucleic amplification, e.g. RT-qPCR or any other method known in the art.

A further object of the invention is a kit for monitoring the acquired immunity of an immunized subject, comprising:
means to detect and/or measure the amount of the biomarker as above described and optionally
control means.

Control means are preferably used to compare the increase of amount of the biomarker to an appropriate control value or amount. The control value or amount may be obtained, for example, with reference to known standard, either from a normal subject or from normal population, preferably from a not immunized or not vaccinated subject.

The means to detect and/or measure the amount of the biomarker as above defined are known to the expert of the art, and are preferably at least one detectably labeled DNA or RNA probe.

The kit of the invention preferably comprises instructions for interpreting the obtained data, e.g. saying that if the amount of said biomarker in the test sample is higher than the control amount, this indicates that the immunized subject is effectively protected or immunized.

In the present invention, the “appropriate control amount” may be the amount quantified, measured or assessed in a sample isolated from a not immunized subject, or from the same subject before immunization. In particular the sample can be isolated from a subject who is not immunized against flu or varicella and/or who has not been vaccinated for flu or varicella. Another example of control group is constituted by patients with liver cirrhosis, individuals in which there is no significant increase in the amount of miR-150 and miR-19b compared to healthy donors.

The biomarker of step a) and the biomarker of step b) of the method of the present invention are preferably the same.

In the present invention, the expression “measuring the amount” can be intended as measuring the amount or concentration or level of the respective miRNA, with any methods known to the skilled in the art. Methods of measuring RNA in samples are known in the art. To measure RNA levels, the cells in a biological sample can be lysed, and the levels of RNA in the lysates or in RNA purified or semi-purified from lysates can be measured by any variety of methods familiar to those in the art. Such methods include hybridization assays using detectably labeled DNA or RNA probes (i.e., Northern blotting), specific acid nucleic amplification, e.g. RT-qPCR, reverse transcription and preamplification, or quantitative or semi-quantitative RT-PCR methodologies using appropriate oligonucleotide primers. Alternatively, quantitative or semi-quantitative in situ hybridization assays can be carried out using, for example, tissue sections, or unlysed cell suspensions, and detectably labeled (e.g., fluorescent, or enzyme-labeled) DNA or RNA probes. Additional methods for quantifying RNA include RNA protection assay (RPA), cDNA and oligonucleotide microarrays, representation difference analysis (RDA), differential display, EST sequence analysis, serial analysis of gene expression (SAGE), quantitative Mass Spectrometry, the massArray platform (Sequenom), and Deep Sequencing and Ion Proton Sequencing Technology.

In the present invention, the expression “detecting” in relation to a nucleic acid, refers to any use of any method of observing, ascertaining or quantifying signals indicating the presence of the target nucleic acid in a sample or the absolute or relative quantity of that target nucleic acid in a sample. Methods can be combined with protein or nucleic acid labeling methods to provide a signal, for example fluorescence, radioactivity, electricity.

miRNAs are present in the bloodstream in a highly stable extracellular form. The existence of distinct circulating populations of miRNAs, associated to either membranous vesicles or protein complexes, may impact the identification of specific miRNAs as reliable markers of disease. Indeed, isolation procedures have been implemented as the first step in the search for such biomarkers, but there is still lack of consensus on the best method for purifying nanovesicles from biological fluids. To address this issue, authors have started from human serum and compared differential centrifugation (Thery, Amigorena et al. 2006) and a new filtration-based nanovesicles purification kit [ExoMiR, Biooscientific] (Bryant, Pawlowski et al. 2012). Authors have found that the two approaches give comparable results.

### FIGURE LEGENDS

The invention will be described in exemplifying examples with reference to the following figures:

FIG. 1. Differential centrifugation versus ExoMir for nanovesicles purification.

Schematic view of two nanovesicle purification methods herein used: differential centrifugation (left) and ExoMir (right). For the latter procedure, serum or cellular medium is passed through 2 filters connected in series. The Top Filter has a larger pore size of approximately 200 nanometers to effectively capture larger particles while the Bottom Filter has a smaller pore size of approximately 20 nanometers for capturing exosomes and other nanovesicles of similar size. The filters are then disconnected and separately flushed by an RNA extraction reagent to lyse the captured particles and release their contents with no preservation of their integrity.

FIG. 2. miRNAs strongly associated with nanovesicles circulating in human serum.

A. Percentage of overlapping results (black, concordant; white, discordant) for ExoMir compared to differential centrifugation for three subpopulations of miRNAs divided by their detectability in differential centrifugation purified nanovesicles, as indicated: undetected (Ct>35) in differential centrifugation samples, detectable (Ct<35) in 3/4 differential centrifugation samples and highly detectable (Ct<31) in 4/4 differential centrifugation samples. B. Venn diagram showing the intersection (22 miRNAs, indicated in the box aside) of miRNAs highly expressed (Ct<31 in all samples) for differential centrifugation (33 total) and ExoMir (30).

FIG. 3. miRNAs compartmentalization in nanovesicles versus soluble fraction circulating in blood of healthy donors.

A. Heatmap for miRNAs significant (p<0.05) upon an ANOVA test (based on F distribution) considering the three reported groups: nanovesicles purified by differential centrifugation; total serum and supernatants from the centrifugation at 110000×g (soluble fraction) from 3 different individuals. Hierarchical clustering was performed considering Log-transformed normalized relative quantities of all coexpressed miRNAs with a Ct<35. Distance: Pearson correlation with complete linkage B. Ranking analysis for miR-150 (left panel) and for miR-19b (right panel) in 10 paired samples of total serum and purified nanovesicles (7 purified by differential centrifugation and 3 by ExoMiR). Lower ranking position=higher representation. C. miR-19b and miR-150 relatives quantities (2̂^{-(specific compartment Ct-total serum Ct)}) by single RT-qPCR assays in nanovesicles compared to soluble fractions from 3 healthy donors sera (mean of the three samples and SEM are reported) processed by differential centrifugation. p value for a 2-way ANOVA analysis showing an extremely significant effect of serum compartmentalization for different miRNAs.

FIG. 4. miR-150 and miR-19b expression in human resting lymphocytes and tissues.

A. Box plot of miRNome relative quantities in 17 different lymphocytic subsets, as indicated (light grey, B lymphocytes; dark grey, CD4 lymphocytes; white, CD8 lymphocytes; black, NK lymphocytes). Only co-expressed miRNAs with a Ct<35 were considered. Dark grey circles indicate miR-150 expression level, light grey circles miR-19b expression level. B. Correlation between miR-19b and miR-150 relative quantities of the 17 lymphocytic subsets was analyzed. Each dot is a distinct lymphocytic subset. Spearman r and p values are reported. C. Expression level of miR-150 and miR-19b in a panel of 20 different human tissues by RT-qPCR, reported as quantities relative to the internal control snRNA U6, hence the data reflect the relative expression among tissues.

FIG. 5. miRNA intracellular modulation and release upon in vitro activation of human lymphocytes.

A. Bio-analyzer qualitative analysis of total RNA extracted 72 h after activation from CD4 lymphocytes (upper panel) and released nanovesicles purified by ExoMir (lower panel). A representative sample is reported. B. Global mean and SEM of miRNome relative quantities of nanovesicle samples (in biological triplicate) released by CD4 lymphocytes upon activation at the indicated time points. Only miRNAs with a Ct<35 at all time points were considered. p value of a Mann-Whitney test comparing 6 h and 96 h is reported. C. Concentration of extracellular IFNgamma revealed in medium modified by CD4 lymphocytes (in biological triplicates) upon activation at the indicated time points. D. Heatmap showing the expression fold change of the indicated miRNAs at the indicated time points upon activation of CD4 lymphocytes compared to Time 0 (T0=1) (left panel); and Log-10 transformed relative expression of the same miRNAs in samples of nanovesicles collected at the indicated time points (right panel). Values are mean of a biological triplicate. The down-regulated (all 5) and the up-regulated (representative 5/56) miRNAs were selected by an ANOVA test (based on F distribution).

FIG. 6. Circulating miR-150 and miR-19b modulation in human serum upon flu vaccination.

A. miRNA quantities relative to exogenous spike-in ath miR-159a in sera of 46 pairs of samples (time of vaccination, T0 and 30 days after, T1) from H1N1-MF59 vaccinated healthy donors. Mean values, SEM and two-tailed paired t-test p value are reported. B. miRNA quantities relative to exogenous spike-in ath miR-159a in sera of 50 H1N1-MF59 vaccinated infants (samples collected at time of first dose, T0, at time of second dose 30 days after, T1 and 30 days after the second dose, T2). Mean values, SEM and two-tailed paired t-test p values are reported. C. Box plot of miR-150 and miR-19b quantities relative to exogenous spike-in ath miR-159a (whiskers: 10-90 percentile) in total serum, purified nanovesicles and purified microvesicles as indicated of 17 pairs of H1N1-MF59 at T0 (white) and T1 (grey). Two-tailed paired t-test p values are reported. D. Log-transformed normalized miRNA quantities in sera of 10 healthy donors and 15 patients affected by liver cirrhosis. Mean values and SEM are reported.

FIG. 7. Circulating miR-150 level correlation to vaccination-associated disease protection.

A. Box plot of indicated miRNA quantities at T1 (30 days after vaccination) relative to exogenous spike-in ath miR-159a (whiskers: 10-90 percentile) in 46 flu vaccinated individuals stratified for having developed an antibody response lower (white) or higher (grey) than 1:320, as assessed by hemagglutination inhibition (HI) titer assay. The p value from a Mann Whitney test is reported. B. Box plot showing the mean fold change of circulating miR-150 upon vaccination of 18 children with Measles-Mumps-Rubella-Varicella stratified for acquisition of protection to varicella.

FIG. 8. Circulating miR-150 modulation in mouse serum upon ovalbumin (OVA) vaccination.

A. miR-150 quantities relative to exogenous spike-in ath miR-159a in serum of either wild type or MHCII^{-/-} mice vaccinated with αGalCer+OVA 2 days before and 7 days after vaccination (each treatment normalized to miR mean relative quantity pre-vaccination). p value for a paired t test is reported. B. Correlation between total Ig concentration (assessed by ELISA) at t=7 days after vaccination in mice vaccinated with αGalCer+OVA (grey dots) or Alum+OVA (white) and serum circulating miR-150 fold change T1/T0 (T1=7 days after vaccination). Spearman r and p values are reported. C. miR-150 intracellular down-regulation (as fold change of expression at 72 hours upon activation in vitro compared to T=0, normalized to the endogenous control smallU6) and extracellular accumulation in purified nanovesicles (EV) at the same time point (72 h) calculated as 2̂-(CtEV-Ctcells)miR-150/2̂-(CtEV-Ctcells)smallU6 for CD4, CD8 and NKT lymphocytes isolated from mouse spleen (CD4 and CD8) and mouse liver (NKT) and activated in vitro as described in Methods.

### EXAMPLES

TABLE I
miR-150 and miR-19b are among the most represented miRNAs
associated with nanovesicles released by human activated
lymphocytes. Representation ranking for the ten most represented
miRNAs associated to nanovesicles released by either CD4 T
helper lymphocytes after 96 hours of activation (left panel) or
B lymphocytes after 24 hours of activation. (right panel).

| CD4 | B |
|---|---|
| miR-150 | miR-299-3p |
| miR-19b | miR-1290 |
| miR-155 | miR-150 |
| miR-223 | miR-875-5p |
| miR-29a | miR-661 |
| miR-222 | miR-223 |
| miR-17 | miR-483-5p |
| miR-625* | miR-601 |
| miR-146a | miR-422a |
| miR-106a | miR-29a |

### Materials and Methods

### Human Samples for Nano-Sized Vesicles Purification

Serum and buffy-coat blood of healthy donors was obtained from the IRCCS Policlinico Ospedale Maggiore in Milano, Italy. The ethical committee of IRCCS Policlinico Ospedale Maggiore in Milano (Italy) approved the use of PBMCs of healthy donors for research purposes and informed consent was obtained from all the subjects involved in this study.

### Vaccination Study Design and Immunogenicity Assessment

Vaccinations to adults were administered at the Dipartimento di Scienze Biomediche per la Salute, University of Milan, Italy, during the month of November 2009. Vaccination to infants (aged 6 to 23 months) were administered in the Department of Maternal and Pediatric Sciences at Fondazione IRCCS Ca' Granda Ospedale Maggiore Policlinico (Milan, Italy) between Nov. 9, 2009, and Jan. 16, 2010 (Esposito, Pugni et al. 2011). Among exclusion criteria for infants there were any treatment in the previous 4 weeks likely to alter their immune response, previous administration of any influenza vaccine and any acute respiratory tract infection in the 4 weeks before enrolment. The studies were approved by the hospital ethics committee, and written informed consent regarding study participation was obtained from all involved adults and the parents or legal guardians of children.

Adults received one dose and children two doses (one month apart) of 0.5 ml of MF59-adjuvanted monovalent 2009 pandemic influenza vaccine (Focetria®, Novartis, Siena, Italy), containing 7.5 μg hemagglutinin of A/California/7/2009(H1N1) (X-181). The vaccine was injected into the deltoid muscle (adults) or into the anterolateral part of the left thigh (infants). Adult serum was collected at time of enrolment (baseline, T0), and 1 month (25±5 days, T1) after vaccination. Infant serum was collected immediately before administration of dose 1 (T0), before administration of dose 2 (28+/−2 days after baseline, T1), and 4 weeks later (56+/−2 days after baseline, T2). 400 μl of serum from both T0 and T1 of 30 vaccinated adults and 200 μl of serum from T0, T1 and T2 of 50 vaccinated children were used to quantify single miRNAs.

In parallel, humoral immune response in both adults and infants was assessed by using the hemagglutination inhibition (HI) test according to standard methods (Menegon, Baldo et al. 1999). This assay determined the antibody titres in serum against the hemagglutinin antigens of the 2009 pandemic influenza strain and the antibody titre was expressed as the reciprocal of the highest dilution that inhibited agglutination.

Infants were vaccinated with GlaxoSmithKline Biologicals' MMRV vaccine Priorix-Tetra™. Study protocols were reviewed and approved by the Research Ethics Committees of the study center involved, and conducted in accordance with the Declaration of Helsinki and the relevant local codes. Written informed consent was obtained from the child's parent or guardian prior to study entry. Blood samples were taken before 38 days after each vaccine dose was administered. Varicella antibodies were measured by immunofluorescence assay (IFA) using a commercially available kit (Virgo™ VZV IgG indirect fluorescent antibody test, Hemagen Diagnostics, MD, USA) with modifications. The cut-off values was an endpoint dilution of 4 dilution-1 for varicella. Seroconversion was defined as the appearance of antibodies at levels greater than or equal to the cut-off value of the relevant assay in subjects seronegative before vaccination. A subject with antibody levels greater than or equal to the cut-off value of the relevant assay was regarded as seropositive.

### Purification of Human and Mouse Lymphocytes and T or B Cell Activation Experiments

Untouched CD4 T helper and B lymphocytes were isolated from human peripheral blood mononuclear cells (PBMC), obtained using Ficoll-Paque on buffy coat of healthy donors, using either CD4 T or B lymphocytes isolation kit (Miltenyi Biotec). CD4 T and B lymphocytes were cultured separately in AIMV medium (devoid of serum, and hence of contaminating miRNAs) and stimulated with either 100 U/ml IL-2, 1 μg/ml PHA (CD4 lymphocytes) or 2.5 μg/ml CpG, 5 μg/ml anti-CD40 (gift of Novartis, Siena, Italy) and 10 μg/ml anti-IgM (BD biosciences) (B lymphocytes). At different time points (6 h, 24 h, 48 h, 72 h and 96 h for CD4 and 24 h for B lymphocytes) cells and conditioned medium were harvested for cell extracts and vesicles isolation (ExoMir).

Liver and spleen were isolated from 4 C57BL/6N mice 8 weeks old. Liver was pressed through 70□ cell strainer (BD). Total liver cells were then resuspended in a 40% Percoll solution. After centrifugation for 20 minutes at 1900 rpm RT without brake, mononuclear cells were isolated in the pellet. After the lysis of red blood cells, mononuclear cells were stained with CD1d tetramer-PE, anti-CD19-FITC and anti-TCR□-APC Abs. A FACS Aria (BD) was used for NKT cell (CD 19⁻, CD1d⁺, TCRb+) sorting. Spleen was pressed through 70□ cell strainer to make single-cell suspension. After the lysis of red blood cells, splenocytes were stained with anti-CD19-FITC, anti-TCR□-PECy7, anti-CD4-PE and anti-CD8-APC Abs. A FACS Aria (BD) was used for CD4⁺ (CD19⁻, TCR□⁺, CD4⁺, CD8⁻) or CD8⁺ (CD19⁻, TCR□⁺, CD4⁻, CD8⁺) T lymphocyte sorting. Purified NKT, CD4⁺, CD8⁺ T lymphocytes were cultured separately in AIMV medium and stimulated with PMA 25 ng/ml, ionomycine 1 □g/ml. Cells were collected for RNA extraction before activation (0 h) and after 72 h of activation. Conditioned medium (72 h) was processed with ExoMir kit for exosomes purification.

### Vesicles Preparation

For differential centrifugation, 2 ml of serum diluted to 4 ml in phosphate buffered saline (PBS) were centrifuged to eliminate floating cells (300×g), dead cells (2,000×g), cellular debris and apoptotic bodies (serum: 12,000×g; cell medium: 10,000×g). The final supernatant was then ultracentrifuged at 110,000×g (100,000×g for cell medium) to pellet the nano-sized vesicles. The pellet was then re-suspended in PBS and filtered through a 0.2 micron filter to eliminate residual larger particles and finally washed in a large volume of PBS, to eliminate contaminating proteins, and centrifuged one last time at the same speed. For double microfiltration (ExoMir, Bio Scientific, Texas) 8 ml of cellular medium or 0.4 ml of human serum diluted to 4 ml with PBS were centrifuged at 300×g and then at 2,000×g. Supernatants were digested by Proteinase K to eliminate proteic complexes and then passed through ExoMir filters. After washing the Top/Bottom filters with 12 ml of PBS (double wash for serum), microvesicles and nano-sized vesicles were separately eluted using 1 ml of BiooPure-MP plus ath-mir-159a (final concentration 3 pM).

miRNAs Profiling and Single miRNA Detection by RT-qPCR

Total RNA from either fresh or frozen human sera; and from either cells or centrifuged vesicular pellets was extracted using miRVana miRNA isolation kit (Ambion), as specified in the protocol, with some modifications. Briefly, 400 μl of thawed serum were mixed with 800 μl of lysis solution composed of RNA Lysis Buffer and synthetic ath-miR-159a (final concentration 2.5 pM). This miRNA was used as process control, for technical normalization. RNA extraction from Top and Bottom Filters (ExoMir) was performed as specified in the protocol and RNA was quantified by Ribogreen (Invitrogen), and characterized by Agilent Bioanalyzer.

3 μl of total RNA were processed for Reverse Transcription and Preamplification with Megaplex Primer Pools A v2.1 and B v2.0 (Applied Biosystems), according to manufacturer instruction. TaqMan Low Density Arrays (Applied Biosystems) were run on a 7900HT Fast Real-Time PCR System. A total of 664 human miRNAs, 6 human small RNA and 1 control miRNA from A. Thaliana were profiled in parallel. Ct values were extracted using RQ Manager, setting a manual threshold of 0.06. For single miRNA detection, a multiplexed Reverse Transcription reaction (up to 5 miRNA) was implemented using the TaqMan miRNA Reverse Transcription Kit and miRNA-specific stem-loop primers (Applied Biosystems) according to manufacturer instruction. To profile miRNA expression in human tissues or cultured cells, 10 ng of RNA were processed for RT (FirstChoice Human Total RNA Survey Panel, Ambion). DCt values were obtained using the Ct of snRNA U6 as endogenous control.

Healthy donors serum samples and serum purified nano-sized vesicles samples were also profiled for 742 miRNAs by using miRNA Ready-to-Use PCR, Human Panel I+II, V2.M qRT-PCR arrays (Exiqon). Normalized values were obtained using a normalization factor resulting from the geometric mean of all expressed miRNAs per sample, i.e. the mean obtained omitting detectors whose Ct is undetermined (Ct>35).

### Mice Studies

MHC II−/− (B6.129-H2Ab1tm1Doi/DoiOrl), C57BL6N (Charles River Italy), were maintained in specific pathogen-free conditions and used at 8 weeks of age. All animal procedures were reviewed and approved by the Institutional Animal Care and Use Committee at San Raffaele Scientific Institute. Following collection of pre-immunization sera, groups of 4 mice were primed at day 0 by subcutaneously (in the left flank) injection of 100 μg/dose of Ovalbumin protein (Sigma) mixed either with 0.1 μg/dose of αGalCer (Alexis), or with Imject Alum Adjuvant (Pierce, Thermo Scientific). Blood was then drawn by retro-orbital phlebotomy after 7 and 14 days to determine specific Ig titers of the primary responses on sera. For measurement of Ag-Specific Ab Titers, individual sera were titrated in parallel at the same time by ELISA. Ab titers are expressed as reciprocal dilutions giving an OD450>mean blank OD450+3 SD. Furthermore, for measurement of circulating miRNAs, 50 microliters of sera of pre-vaccinated and 7 and 14 days post-vaccinated mice were processed for total RNA extraction and miR-150 was quantified by single TaqMan assays.

### Results

Identification of a Robust Signature of miRNAs Associated with Nano-Sized Vesicles Circulating in Blood of Healthy Donors.

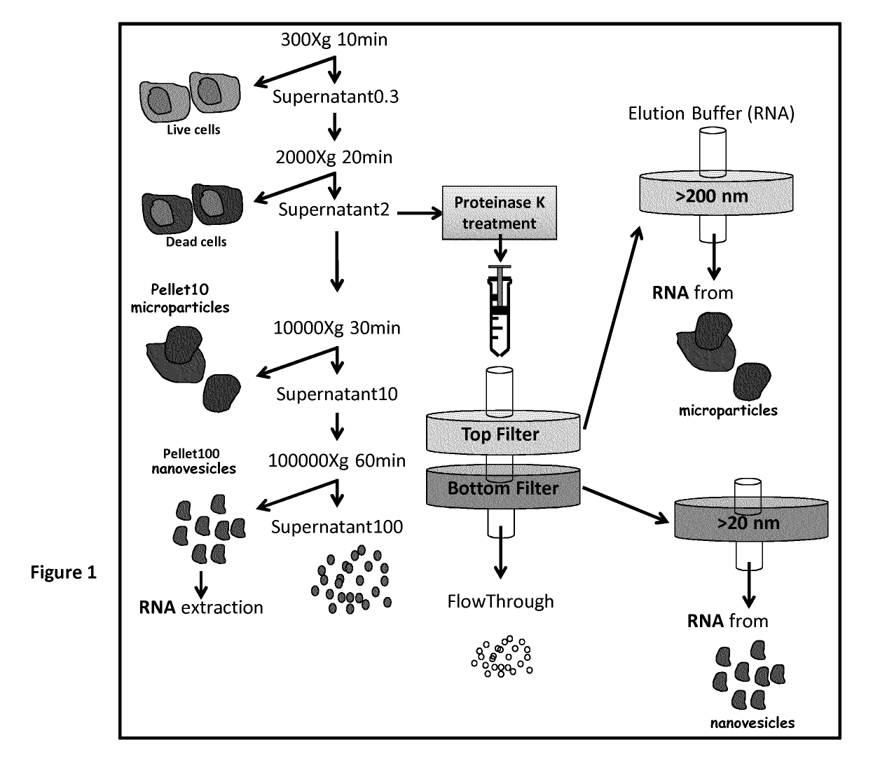

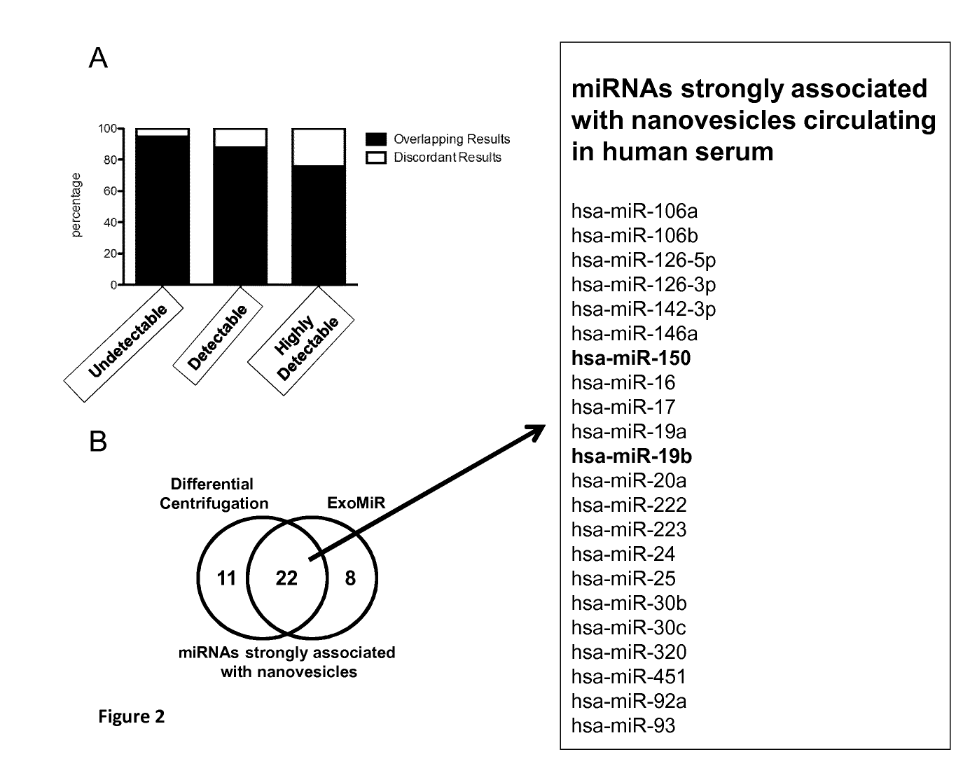

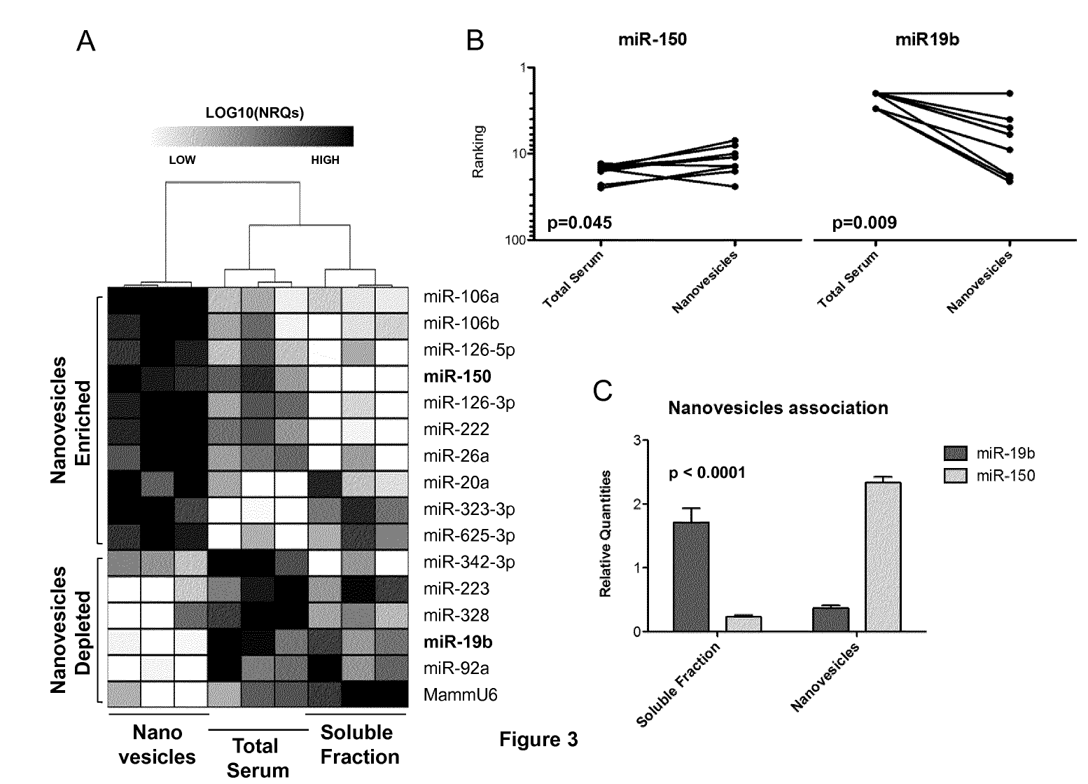

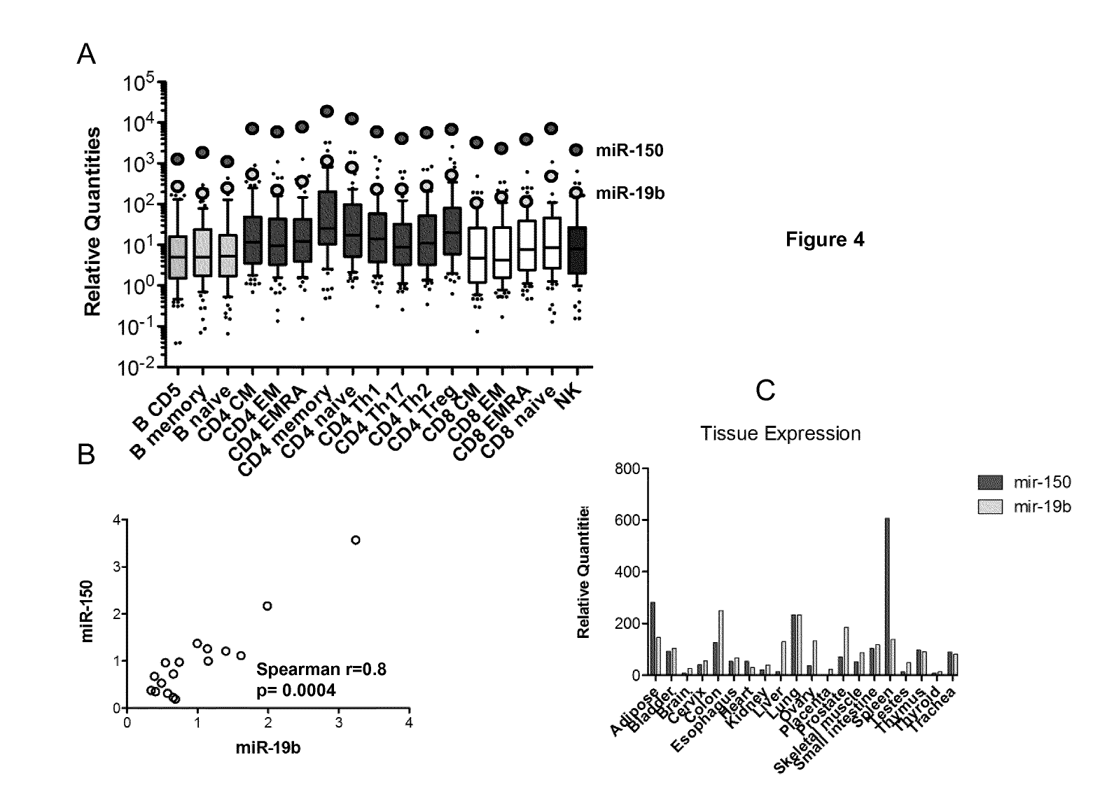

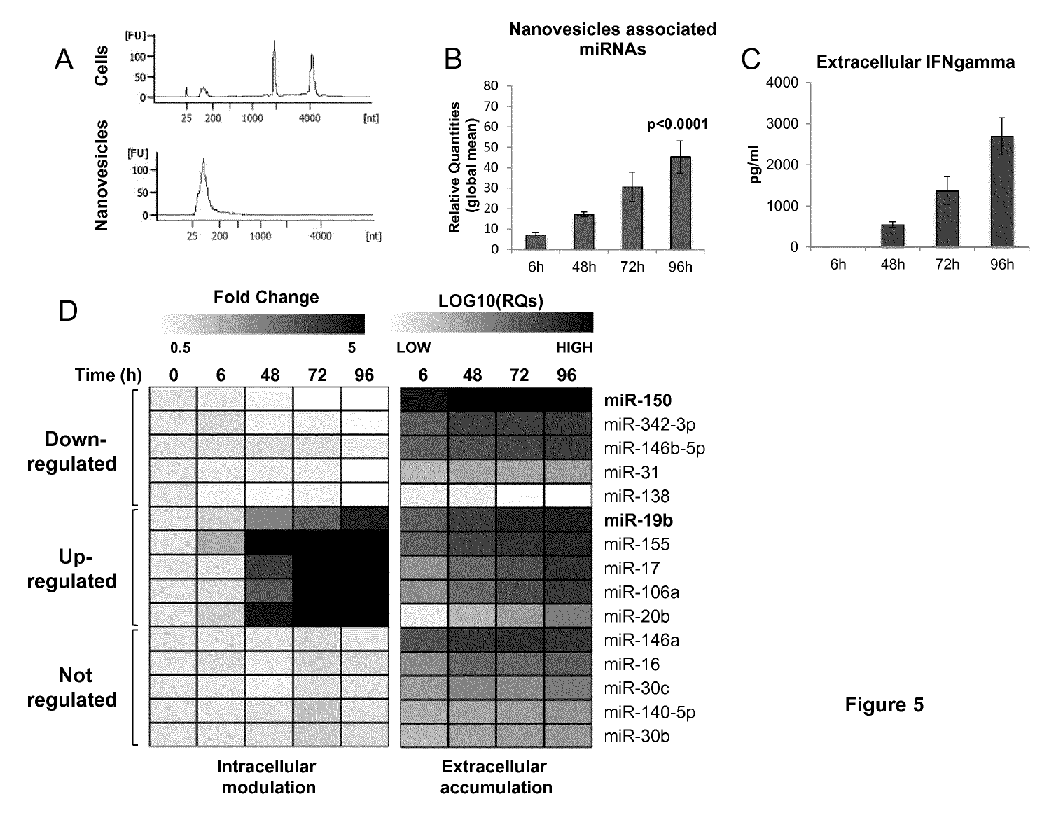

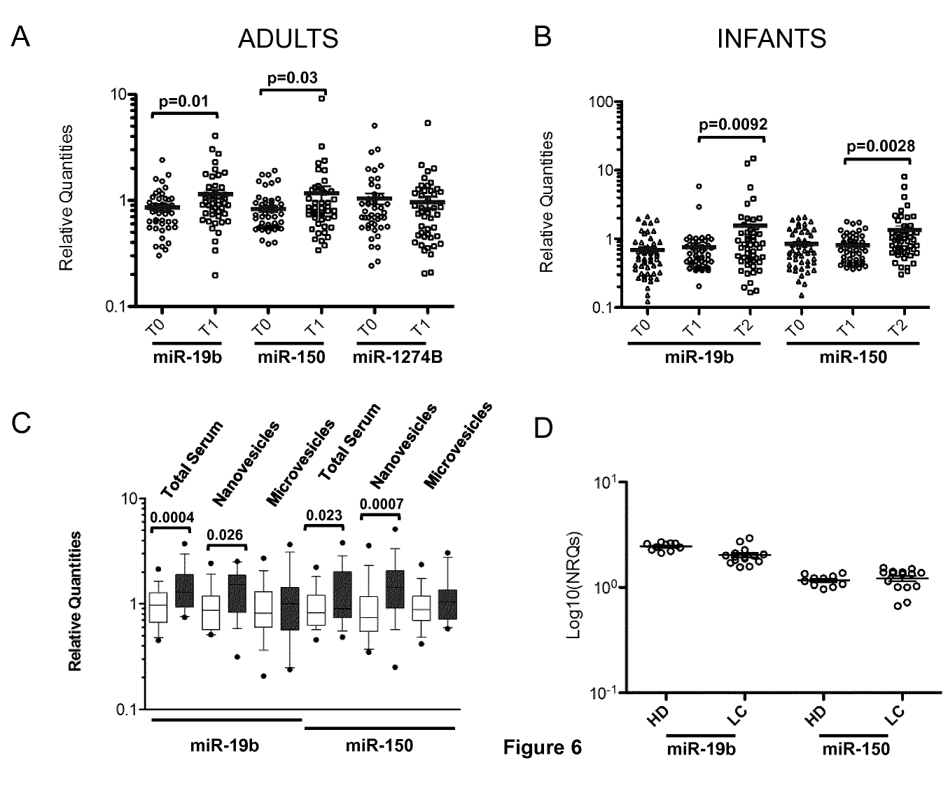

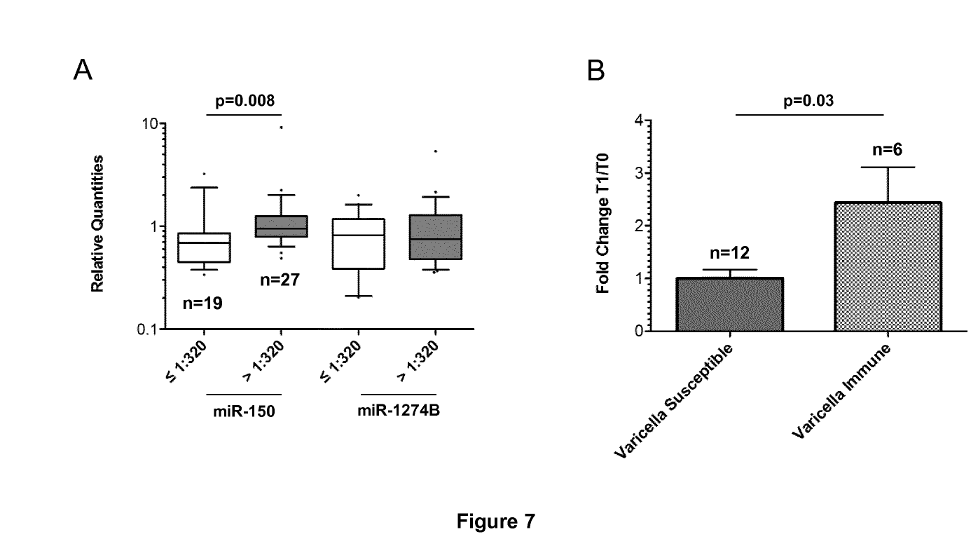

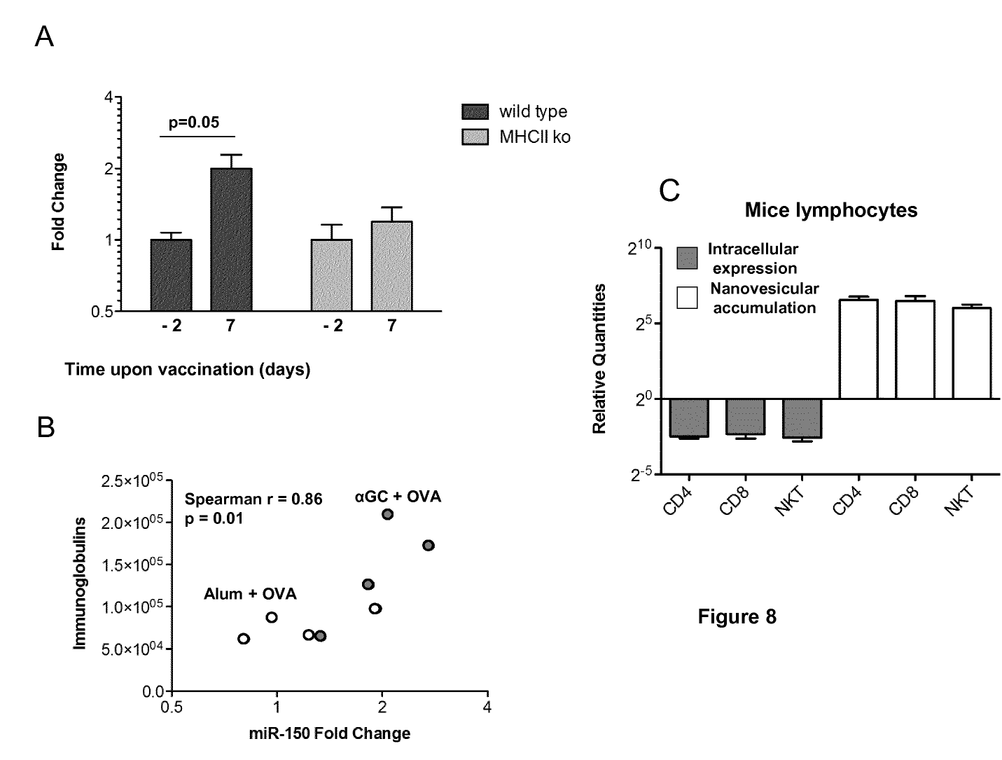

In order to characterize a signature of miRNAs strongly associated with nano-sized vesicles (nanovesicles) circulating in human blood, authors purified them by differential centrifugation from serum of three healthy donors and by ExoMir™ kit (Biooscientific, Texas, USA) from three additional healthy donors. While the process of purification by differential centrifugation has been already described in detail (Thery, Amigorena et al. 2006), ExoMir is an alternative method based on microfiltration (Bryant, Pawlowski et al. 2012). The general workflow for both methods is depicted in FIG. 1. The miRNome from either total serum or nanovesicles was profiled by Reverse Transcriptase quantitative PCR (RT-qPCR) using TaqMan Low Density Arrays (TLDA, Applied Biosystems). In order to establish if ExoMir purification technique was indeed reproducing results obtained by differential centrifugation in terms of nanovesicle miRNA representation, authors analyzed the percentage of overlapping results for three groups of miRNAs. The first group was composed of miRNAs that were undetected in differential centrifugation samples and 94.7% of these miRNAs were also showing a Ct>35 in at least 2/3 ExoMir samples. The second group was composed of miRNAs that were detectable in differential centrifugation samples with a Ct<35 (detectable miRNAs) and 87.9% of these miRNAs were also showing a Ct<35 in at least 2/3 ExoMir samples. The third group was composed of miRNAs that were strongly represented in differential centrifugation samples, being detected in 4/4 samples with a Ct<31 (highly detectable miRNAs). 75.9% of these miRNAs were similarly detected in 3/3 ExoMir samples with a Ct<31 (FIG. 2A).

By intersecting results of the two purification strategies, authors obtained a list of 22 miRNAs that can be regarded as strongly associated with circulating nanovesicles, for being robustly expressed in all purified samples, independently of the purification method (FIG. 2B). To analyze in greater detail the distribution of specific miRNAs in different serum compartments, authors evaluated miRNA expression in purified nanovesicles, total serum and in the supernatant of the centrifugation at 110000×g (soluble fraction) for three healthy donors. Hierarchical clustering analysis showed that nanovesicle-associated miRNome is more distant to samples of total serum and soluble fraction, suggesting a specific miRNA quantitative pattern for isolated nanovesicles (FIG. 3A). A one-way ANOVA analysis revealed the existence of two distinct families of miRNAs: the ones that are enriched in nanovesicles and the ones with the opposite behavior being more represented in total serum and soluble fraction samples (FIG. 3A). Then miR-150 and miR-19b, key regulators of lymphocyte differentiation and functions, are part of the signature of miRNAs strongly associated with nanovesicles circulating in human serum. Furthermore they showed opposite behaviour in terms of specific enrichment in nanovesicles compared to total serum and soluble fraction. More specifically, while miR-150 was enriched quantitatively when purifying nanovesicles, miR-19b showed a higher level in total serum or soluble fraction than in isolated nanovesicles. The preferential association with or depletion from nanovesicles was then confirmed by ranking analysis and RT-qPCR single assays using sera from additional donors (FIG. 3B-C).

miR-150 and miR-19b Expression in Human Lymphocytes and Representation in Nanovesicles Released by Lymphocytes Upon Activation.

miR-150 and miR-19b were found to be among the most highly expressed miRNA in 17 different lymphocyte subsets purified from peripheral blood mononuclear cells of healthy donors [(FIG. 4A and (Rossi, Rossetti et al. 2011)]. Their expression level in different lymphocyte populations was found to be extremely concordant, showing the highest expression in CD4 lymphocytes. Moreover, miR-150 (but not miR-19b) was also found to be highly abundant in spleen tissue compared to other tissues (FIG. 4C).

To specifically characterize the miRNome associated with nanovesicles released in the extracellular milieu by human lymphocytes upon in vitro activation, ex vivo isolated resting CD4 cells were stimulated with 100 U/ml IL-2 and 1 μg/ml PHA; at different time points upon activation (6, 48, 72 and 96 hours), extracellular nanovesicles were purified by ExoMir. Qualitative analysis of total RNA showed a significant enrichment of small RNA molecules in purified nanovesicles compared to cellular RNA (FIG. 5A). Moreover, similarly to INF-γ extracellular increment, the global mean of miRNA relative expression (profiled by TLDA) associated with extracellular nanovesicles dramatically increased over time (FIG. 5B-C). For the majority of miRNAs, the extracellular accumulation was paralleled by either no intracellular modulation, or a significant up-regulation, as in the case of miR-155 and miR-19b (FIG. 5D). Differently, miR-150 was part of a very small group of miRNAs (miR-150, miR-342-3p, miR-146b-5p and miR-31) whose extracellular accumulation was paralleled by a specific intracellular down-modulation upon activation (FIG. 5D). Importantly, miR-150 and miR-19b resulted to be the most represented miRNAs associated with nanovesicles purified in the extracellular milieu of stimulated CD4 lymphocytes (Table I). Moreover, when ex vivo isolated resting B cells were stimulated with 2.5 μg/ml CpG, 5 μg/ml anti-CD40 and 10 μg/ml anti-IgM; and extracellular nanovesicles purified by ExoMir, miR-150 was also among the most represented miRNAs associated with nanovesicles purified in the extracellular milieu (Table I).

Human Serum Circulating miR-150 and miR-19b do Increase Upon Vaccination.

Having observed that activated lymphocytes, at least in vitro, release highly abundant quantity of miR-150 and miR-19b, and that these miRNAs are easily detectable in human serum in resting conditions, authors were prompted to evaluate if the level of serum circulating miR-150 and miR-19b would be sensibly modulated upon vaccine administration and activation of the immune system.

To this aim, authors analyzed serum samples from 46 healthy adults and 50 infants vaccinated with H1N1 MF59 for miR-150 and miR-19b relative quantity by RT-qPCR. For each donor, authors had serum collected at time 0 of vaccination (T0) and at time 30 days after vaccination (T1). For infants, who were administered a second dose of vaccine at T1, authors had also serum collected 30 days after the boost (T2). While miR-1274B, which was strongly associated with vesicles released not only by lymphocytes but also by non-lymphoid cells (data not shown), failed to show any modulation in total serum of vaccinated adults, miR-150 and miR-19b did indeed increase in sera of post-vaccinees, as hypothesized (FIG. 6A). Neither age nor gender of vaccinated individuals impacted miR relative quantity post-vaccination and T1/T0 fold change (data not shown). In infants who had never encountered influenza virus before vaccination, miR-150 and miR-19b level was not modulated 30 days after the first dose of vaccine (T1) but significantly increased 30 days after the second dose (T2) (FIG. 6B).

To analyze if the increase of miR-150 and miR-19b was specifically compartmentalized in serum circulating nanovesicles, serum samples from 17 adults vaccinated with H1N1 MF59 (T0 and T1 as above) were used to purify both nanovesicles and vesicles of larger size (>200 nanometers, here called microvesicles) by ExoMir. Circulating miR-150 increase upon vaccination was highly significant and more evident in isolated nanovesicles compared to total serum and it was not registered in isolated microvesicles (FIG. 6C), suggesting a specific process of miR-150 release through nanovesicles during immune response. For miR-19b, authors observed that it increased in the nanovesicular fraction and not in the microvesicular one, but that, differently than miR-150, miR-19b increased more evidently in total serum than in purified nanovesicles, and hence that the purification of nanovesicles did not improve the detection of the phenomenon. The increase in circulating miR-150 and miR-19b is not an aspecific phenomenon related to different types of physiological or pathological conditions, as suggested by the fact that they were not modulated in patients affected by liver cirrhosis as compared to healthy donors (FIG. 6D).

Importantly, in flu vaccinated adults, miR-150 relative quantity registered at T1 was found to be significantly higher in individuals mounting higher antibody response (as surveyed by a Hemagglutinin Inhibition (HI) titer assay) (FIG. 7A).

To evaluate if the correlation of circulating miR-150 level and antibody response was also true in case of different vaccinations than flu, authors analyzed measles-mumps-rubella-varicella (MMRV) vaccinated infants. They had sera collected at time of vaccination (T0) and 34-37 days after (T1). When infants were stratified for having or having not acquired immunity to varicella, it was possible to observe a higher increase of serum miR-150 level upon vaccination in varicella immunized infants compared to varicella susceptible infants (FIG. 7B).

Mouse Serum Circulating miR-150 is Modulated Upon Lymphocyte Activation In Vivo.

Consistently with results in vaccinated individuals, wild type mice vaccinated with OVA adjuvanted with alpha-galactosylceramide (αGalCer), a strong activator of lymphocyte response, showed a tidy increase of serum miR-150, detectable 7 days after vaccination (T1 vs T0, T0 being serum collected two days before vaccination) (FIG. 8A). This increase was still traceable but not significant in wild type mice vaccinated with OVA adjuvanted with Aluminum Hydroxide+Magnesium Hydroxide (Alum), and the increment of circulating miR-150 upon vaccination (expressed as fold change T1/T0) was found to significantly correlate with the level of Immuno-globulins developed against OVA at the same time point (FIG. 8B), demonstrating that it depended on lymphocyte activation in vivo.

To evaluate if circulating miR-150 modulation was affected by specific lymphocyte depletion/impairment, authors also vaccinated MHCII knock out mice, that are depleted of mature CD4 T cells and deficient in cell-mediated immune responses (Grusby, Johnson et al. 1991). Circulating miR-150 increment upon αGalCer OVA vaccination was significantly lower in MHCII ko mice (FIG. 8A).

Moreover, consistently with results in human primary cells, murine T lymphocytes down-regulated miR-150 upon in vitro activation and accumulated it in extracellular nanovesicles (FIG. 8C).

### REFERENCES

Allantaz, F., D. T. Cheng, et al. (2012). “Expression profiling of human immune cell subsets identifies miRNA-mRNA regulatory relationships correlated with cell type specific expression.” PLoS One 7(1): e29979.
Arroyo, J. D., J. R. Chevillet, et al. (2011). “Argonaute2 complexes carry a population of circulating microRNAs independent of vesicles in human plasma.” Proc Natl Acad Sci USA 108(12): 5003-5008.
Bala, S., J. Petrasek, et al. (2012). “Circulating microRNAs in exosomes indicate hepatocyte injury and inflammation in alcoholic, drug-induced and inflammatory liver diseases.” Hepatology.
Bartel, D. P. (2004). “MicroRNAs: genomics, biogenesis, mechanism, and function.” Cell 116(2): 281-297.
Bender, T. P., C. S. Kremer, et al. (2004). “Critical functions for c-Myb at three checkpoints during thymocyte development.” Nat Immunol 5(7): 721-729.
Bezman, N. A., T. Chakraborty, et al. (2011). “miR-150 regulates the development of NK and iNKT cells.” J Exp Med 208(13): 2717-2731.
Bryant, R. J., T. Pawlowski, et al. (2012). “Changes in circulating microRNA levels associated with prostate cancer.” Br J Cancer 106(4): 768-774.
Chen, H., Y. Wang, et al. (2009). “Serologic survey of pandemic (H1N1) 2009 virus, Guangxi Province, China.” Emerg Infect Dis 15(11): 1849-1850.
Chen, X., Y. Ba, et al. (2008). “Characterization of microRNAs in serum: a novel class of biomarkers for diagnosis of cancer and other diseases.” Cell Res 18(10): 997-1006.
Chim, S. S., T. K. Shing, et al. (2008). “Detection and characterization of placental microRNAs in maternal plasma.” Clin Chem 54(3): 482-490.
Cortez, M. A., C. Bueso-Ramos, et al. (2011). “MicroRNAs in body fluids—the mix of hormones and biomarkers.” Nat Rev Clin Oncol 8(8): 467-477.
Esposito, S., L. Pugni, et al. (2011). “Influenza A/H1N1 MF59-adjuvanted vaccine in preterm and term children aged 6 to 23 months.” Pediatrics 127(5): e1161-1168.
Friedman, R. C., K. K. Farh, et al. (2009). “Most mammalian mRNAs are conserved targets of microRNAs.” Genome Res 19(1): 92-105.
Ghisi, M., A. Corradin, et al. (2011). “Modulation of microRNA expression in human T-cell development: targeting of NOTCH3 by miR-150.” Blood 117(26): 7053-7062.
Grusby, M. J., R. S. Johnson, et al. (1991). “Depletion of CD4+ T cells in major histocompatibility complex class II-deficient mice.” Science 253(5026): 1417-1420.
Jopling, C. L., M. Yi, et al. (2005). “Modulation of hepatitis C virus RNA abundance by a liver-specific MicroRNA.” Science 309(5740): 1577-1581.
Kawabe, T., T. Naka, et al. (1994). “The immune responses in CD40-deficient mice: impaired immunoglobulin class switching and germinal center formation.” Immunity 1(3): 167-178.
Lotvall, J. and H. Valadi (2007). “Cell to cell signalling via exosomes through esRNA.” Cell Adh Migr 1(3): 156-158.
Menegon, T., V. Baldo, et al. (1999). “Influenza vaccines: antibody responses to split virus and MF59-adjuvanted subunit virus in an adult population.” Eur J Epidemiol 15(6): 573-576.
Mitchell, P. S., R. K. Parkin, et al. (2008). “Circulating microRNAs as stable blood-based markers for cancer detection.” Proc Natl Acad Sci USA 105(30): 10513-10518.
Monticelli, S., K. M. Ansel, et al. (2005). “MicroRNA profiling of the murine hematopoietic system.” Genome Biol 6(8): R71.
O'Connell, R. M., K. D. Taganov, et al. (2007). “MicroRNA-155 is induced during the macrophage inflammatory response.” Proc Natl Acad Sci USA 104(5): 1604-1609.
Raposo, G., H. W. Nijman, et al. (1996). “B lymphocytes secrete antigen-presenting vesicles.” J Exp Med 183(3): 1161-1172.
Reid, G., M. B. Kirschner, et al. (2011). “Circulating microRNAs: Association with disease and potential use as biomarkers.” Crit Rev Oncol Hematol 80(2): 193-208.
Rossi, R. L., G. Rossetti, et al. (2011). “Distinct microRNA signatures in human lymphocyte subsets and enforcement of the naive state in CD4+ T cells by the microRNA miR-125b.” Nat Immunol 12(8): 796-803.
Salomoni, P., D. Perrotti, et al. (1997). “Resistance to apoptosis in CTLL-2 cells constitutively expressing c-Myb is associated with induction of BCL-2 expression and Myb-dependent regulation of bc1-2 promoter activity.” Proc Natl Acad Sci USA 94(7): 3296-3301.
Schopman, N. C., S. Heynen, et al. (2010). “A miRNA-tRNA mix-up: tRNA origin of proposed miRNA.” RNA Biol 7(5): 573-576.
Simons, M. and G. Raposo (2009). “Exosomes—vesicular carriers for intercellular communication.” Curr Opin Cell Biol 21(4): 575-581.
Starkey Lewis, P. J., J. Dear, et al. (2011). “Circulating microRNAs as potential markers of human drug-induced liver injury.” Hepatology 54(5): 1767-1776.
Thery, C., S. Amigorena, et al. (2006). “Isolation and characterization of exosomes from cell culture supernatants and biological fluids.” Curr Protoc Cell Biol Chapter 3: Unit 3 22.
Thery, C., M. Ostrowski, et al. (2009). “Membrane vesicles as conveyors of immune responses.” Nat Rev Immunol 9(8): 581-593.
Thomas, M. D., C. S. Kremer, et al. (2005). “c-Myb is critical for B cell development and maintenance of follicular B cells.” Immunity 23(3): 275-286.
Valadi, H., K. Ekstrom, et al. (2007). “Exosome-mediated transfer of mRNAs and microRNAs is a novel mechanism of genetic exchange between cells.” Nat Cell Biol 9(6): 654-659.
Vesikari, T., M. Pellegrini, et al. (2009). “Enhanced immunogenicity of seasonal influenza vaccines in young children using MF59 adjuvant.” Pediatr Infect Dis J 28(7): 563-571.
Volinia, S., G. A. Calin, et al. (2006). “A microRNA expression signature of human solid tumors defines cancer gene targets.” Proc Natl Acad Sci USA 103(7): 2257-2261.
Wang, J. F., M. L. Yu, et al. (2010). “Serum miR-146a and miR-223 as potential new biomarkers for sepsis.” Biochem Biophys Res Commun 394(1): 184-188.
Xiao, C., D. P. Calado, et al. (2007). “MiR-150 controls B cell differentiation by targeting the transcription factor c-Myb.” Cell 131(1): 146-159.
Zheng, Q., L. Zhou, et al. (2012). “MicroRNA miR-150 is involved in Valpha14 invariant NKT cell development and function.” J Immunol 188(5): 2118-2126.
Zhou, B., S. Wang, et al. (2007). “miR-150, a microRNA expressed in mature B and T cells, blocks early B cell development when expressed prematurely.” Proc Natl Acad Sci USA 104(17): 7080-7085.

1-8. (canceled)

9. An in vitro method of monitoring the acquired immunity of an immunized subject comprising the following steps:
a) obtaining a biological sample from the immunized subject,
b) measuring the amount of a biomarker consisting of at least one miRNA selected from the group consisting of miR-150 (SEQ ID NO: 1) and miR-19b (SEQ ID NO: 2), present in the biological sample, and
c) comparing the measured amount of step b) with an appropriate control amount of said biomarker, wherein if the amount of said biomarker in the biological sample is higher than the control amount, this indicates that the immunized subject is effectively protected.

10. The in vitro method of claim 9, wherein the subject is an adult or a child.

11. The in vitro method according to claim 10, wherein the biological sample is blood, serum or cellular medium of ex vivo cultured cells of the immunized subject.

12. The in vitro method of claim 9 wherein the method further comprises isolating nanovesicles from the biological sample.

13. The in vitro method according to claim 9, wherein the acquired immunity is due to a vaccination.

14. The in vitro method according to claim 13, wherein the vaccination is a flu or varicella vaccination.

15. The in vitro method according to claim 14, wherein the flu vaccination is performed with the H1N1 MF59 vaccine.

16. The in vitro method according to claim 14, wherein the varicella vaccination is performed with Measles-Mumps-Rubella-Varicella vaccine.

17. The in vitro method according to claim 9, wherein the amount of the biomarker is measured by specific acid nucleic amplification.

18. A kit for monitoring the acquired immunity of an immunized subject, comprising:
means to detect and/or measure; a biomarker consisting of at least one miRNA selected from the group consisting of miR-150 (SEQ ID NO: 1) and miR-19b (SEQ ID NO: 2),
and optionally
control means.

19. The in vitro method of claim 12 wherein the nanovesicles are isolated from the cellular medium of ex vivo cultured cells of the immunized subject.

20. The in vitro method of claim 19 wherein the ex vivo cultured cells of the immunized subject are T or B lymphocytes.

21. The in vitro method of claim 20 wherein the method further comprises activating the T or B lymphocytes prior to step b).

22. The in vitro method of claim 12 wherein the nanovesicles are isolated by differential centrifugation or by microfiltration.

23. The in vitro method of claim 12 wherein the nanovesicles range in diameter from 20 to 200 nanometers.


SEQUENCE LISTING (2 sequences)

SEQ ID NO: 1 - RNA, 22 nt, Homo sapiens
ucucccaacccuuguaccagug

SEQ ID NO: 2 - RNA, 23 nt, Homo sapiens
ugugcaaauccaugcaaaacuga